(19) United States
(12) Patent Application Publication
(10) Pub. No.: US 2024/0257392 A1
(43) Pub. Date: Aug. 1, 2024

(54) Fall Detection and Prevention System for Alzheimer's, Dementia, and Diabetes

(71) Applicant: Arizona Board of Regents on Behalf of the University of Arizona, Tucson, AZ (US)

(72) Inventors: Kavan Hazeli, Tucson, AZ (US); Bahram Jalali, Los Angeles, CA (US); Xuejing Wang, Tucson, AZ (US); Janet Roveda, Tucson, AZ (US)

(21) Appl. No.: 18/429,089

(22) Filed: Jan. 31, 2024

Related U.S. Application Data
(60) Provisional application No. 63/482,345, filed on Jan. 31, 2023.
(60) Provisional application No. 63/499,073, filed on Apr. 28, 2023.
(60) Provisional application No. 63/548,043, filed on Nov. 10, 2023.

(51) Int. Cl. G06T 7/73; A61B 5/00; A61B 5/107; G06T 7/12; G06T 7/194; G06T 7/55; G06T 7/66; G08B 21/04; G16H 10/60; G16H 40/67

(52) U.S. Cl. CPC G06T 7/74; A61B 5/1077; A61B 5/1079; A61B 5/746; G06T 7/12; G06T 7/194; G06T 7/55; G06T 7/66; G08B 21/043; G08B 21/0476; G16H 10/60; G16H 40/67; G06T 2207/10016; G06T 2207/10028; G06T 2207/20081; G06T 2207/20084; G06T 2207/30004; G06T 2207/30196

(57) ABSTRACT

A fall prevention system that monitors the real-time pose of a user and provides alerts in response to a determination that the user may be likely to fall. To accurately determine whether the user is in an unstable pose, the fall prevention system receives video images of the user (and, in some instances, depth information) captured by multiple image capture systems from multiple angles. To process multiple video streams with sufficient speed to provide alerts in near real-time, the fall prevention system uses a pose estimation and stability evaluation process that is optimized to reduce computational expense. For example, the fall prevention process may be realized by a local controller (e.g., worn by the user) that receives video images via a local connection and processes those images locally using pre-trained machine learning models that are uniquely capable of quickly capturing and evaluating the pose of the user.

### CROSS-REFERENCE TO RELATED APPLICATIONS

This application claims priority to U.S. Prov. Pat. Appl. No. 63/482,345, filed Jan. 31, 2023, U.S. Prov. Pat. Appl. No. 63/499,073, filed Apr. 28, 2023, and U.S. Prov. Pat. Appl. No. 63/548,043, filed Nov. 10, 2023. Additionally, some embodiments of the disclosed technology can be used with some of the embodiments described in U.S. Prov. Pat. Appl. No. 63/399,901, filed Aug. 22, 2022, U.S. patent application Ser. No. 18/236,842, filed Aug. 22, 2023, U.S. Prov. Pat. Appl. No. 63/383,997, filed Nov. 16, 2022, and U.S. patent application Ser. No. 18/511,736, filed Nov. 16, 2023. Each of those applications are hereby incorporated by reference.

### FEDERAL FUNDING

None

### BACKGROUND

Falls are a complex, multifactorial issue that leads to high morbidity, hospitalization rate, and mortality in the elderly population. Falls and associated outcomes harm the injured individuals and affect their families, friends, care providers, and strain the public health system. While all elderly individuals are at risk, people with Alzheimer's disease or dementia fall more often compared to cognitively healthy older adults. Falls affect between 60 to 80 percent of individuals with cognitive impairment. Individuals with dementia are up to three times more likely to sustain a hip fracture compared to cognitively intact older adults. Some of the most common factors that have contributed to falls are changes in gait and balance, changes in visual perception, and confusion and delirium.

An estimated 34.2 million people have diabetes—approximately 10.5 percent of the U.S. population. Diabetes is a systemic disease as it affects various body systems to some extent. Strong evidence has been reported that diabetes mellitus enhances the threat of cognitive impairment, dementia, and changes in visual perception. Diabetes patients, who have a 10 to 30 times higher lifetime chance of having a lower extremity amputation (LEA) than the general population, frequently sustain injuries due to changes in their visual perception thus colliding with stationary objects. In one to three years, 20 to 50 percent of diabetic amputees will reportedly need to amputate their second limb, and more than 50 percent will do so in five years.

A number of prior art systems assess the severity of falls to determine the likelihood of a potential injury. However, there is a need for a system that provides alerts in real time to prevent falls before they occur.

### SUMMARY

To overcome those and other drawbacks in the prior art, a fall prevention system is disclosed that monitors the real-time pose of a user and provides alerts in response to a determination that the user may be likely to fall. To accurately determine whether the user is in an unstable pose, the fall prevention system receives video images of the user (and, in some instances, depth information) captured by multiple image capture systems from multiple angles. To process multiple video streams with sufficient speed to provide alerts in near real-time, the fall prevention system uses a pose estimation and stability evaluation process that is optimized to reduce computational expense. For example, the fall prevention process may be realized by a local controller (e.g., worn by the user) that receives video images via a local connection and processes those images locally using pre-trained machine learning models that are uniquely capable of quickly capturing and evaluating the pose of the user.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1A is a diagram of an architecture of a fall prevention system according to exemplary embodiments.

FIG. 1B is a block diagram of the architecture of FIG. 1A according to exemplary embodiments.

FIG. 2 is a block diagram of a fall prevention process according to exemplary embodiments.

FIG. 3 are diagrams illustrating the human contour of a user undergoing an unbalancing process leading to falling.

FIG. 4 is a block diagram of various pose estimation processes according to exemplary embodiments.

FIG. 5A illustrates an example original image divided into an N×N grid.

FIG. 5B illustrates an example of a rectangular-shaped bounding box highlighting an object in the original image of FIG. 5A.

FIG. 5C illustrates Intersection Over Unions (IOU), a parameter for distinguishing grids highly relevant to the objects from lowly ones.

FIG. 5D illustrates the use of Non-Maximum Suppression (NMS) to keep the bounding boxes with the highest probability scores.

FIG. 6A is a diagram of landmarks generated by a pose detection process according to exemplary embodiments.

FIG. 6B is an example image with the landmarks of FIG. 6A.

FIG. 6C is an example segmentation mask identified based on the example image of FIG. 6B and the landmarks of FIG. 6A.

FIG. 7 is a block diagram of various stability evaluation processes according to exemplary embodiments.

FIG. 8 is an illustration of example course stability evaluations according to an exemplary embodiment.

FIG. 9 is a diagram of stability metrics generated using the human contour of a stable user and an unstable user according to exemplary embodiments.

FIG. 10 is a diagram illustrating a process for estimating the center of mass of a user according to exemplary embodiments.

FIG. 11 illustrates a skewness calculation according to exemplary embodiments.

FIG. 12 illustrates an example of how the use of multiple image capture systems can more accurately determine whether the user is likely to fall.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

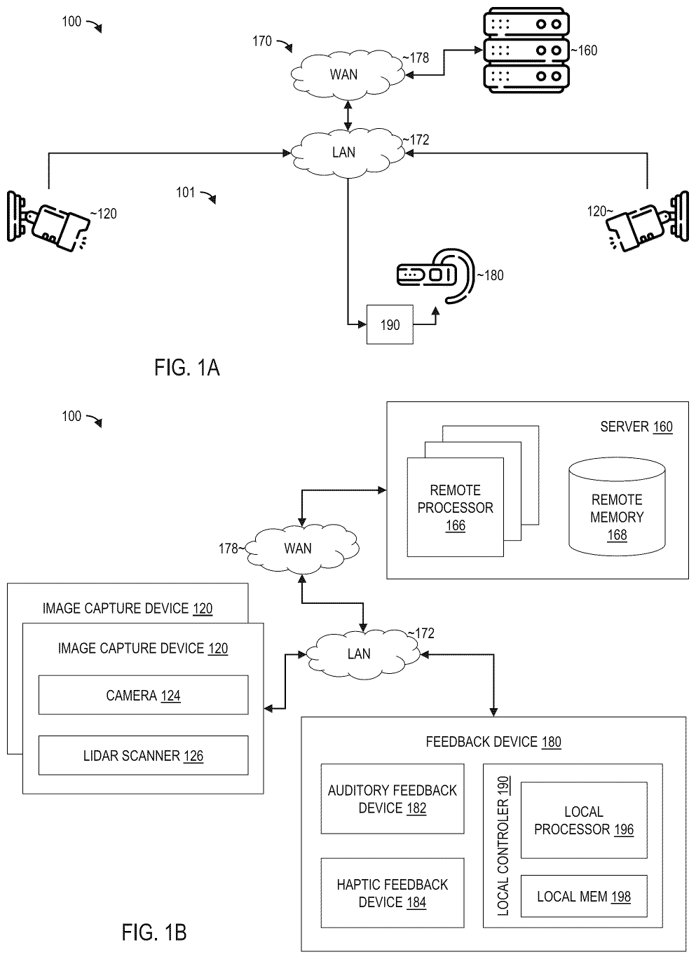

FIGS. 1A and 1B are diagrams of an architecture 100 of a fall prevention system according to exemplary embodiments.

In the embodiment of FIG. 1A, the architecture 100 includes multiple image capture systems 120 in communication with a local controller 190 and a feedback device 180 via one or more communication networks 170 (e.g., a local area network 172). In some embodiments, the local controller 190 (and/or the feedback device 180 and/or the image capture systems 120) may communicate with a remote server 160 via a wide area network 178 (e.g., the internet).

As shown in FIG. 1B, feedback device 180 includes an auditory feedback device 182 (e.g., a speaker). In some embodiments, the feedback device 180 may also include a haptic feedback device 184 (for example, as described in U.S. patent application Ser. No. 18/236,842). The server 160 may include one or more hardware computer processing units (remote processor(s) 166) and non-transitory computer readable storage media (remote memory 168).

The local controller 190 may be any hardware computing device suitably configured to perform the functions described herein. As shown in FIG. 1B, the local controller 190 may include a hardware computer processing unit (local processor 196) and non-transitory computer readable storage media (local memory 198). As described in more detail below, the local controller 190 may be integrated with the feedback device 180 as shown in FIG. 1B or may be realized as a separate device that communicates with the feedback device 180 via a wired or wireless connection (e.g., Bluetooth, WiFi, etc.).

Each image capture device 120 includes a camera 124 that capture two-dimensional video images of the environment 101 of the user. In preferred embodiments, each image capture device 120 also captures depth information from the environment 101. Accordingly, in those embodiments, the camera 124 may be a depth sensing camera (e.g., a stereoscopic camera). Alternatively, as shown in FIG. 1B, each image capture device 120 may include both a camera 124 and a light detection and ranging (LiDAR) scanner 126.

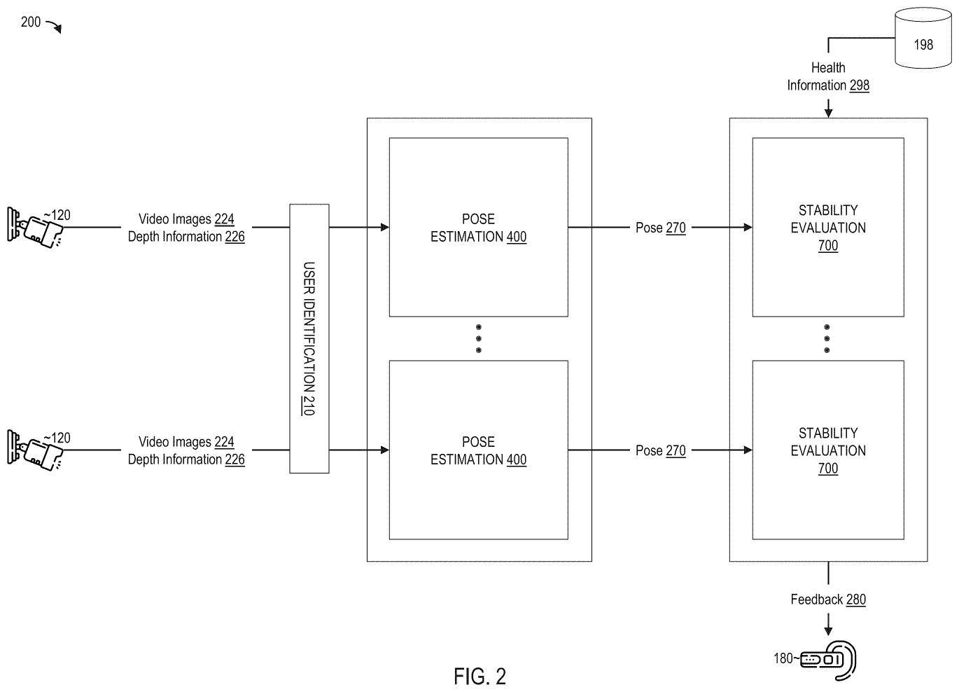

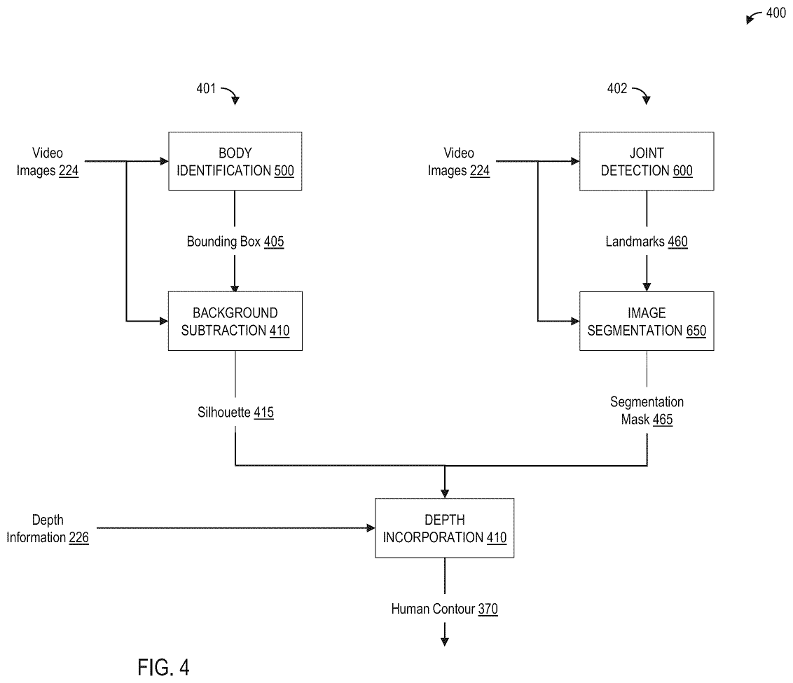

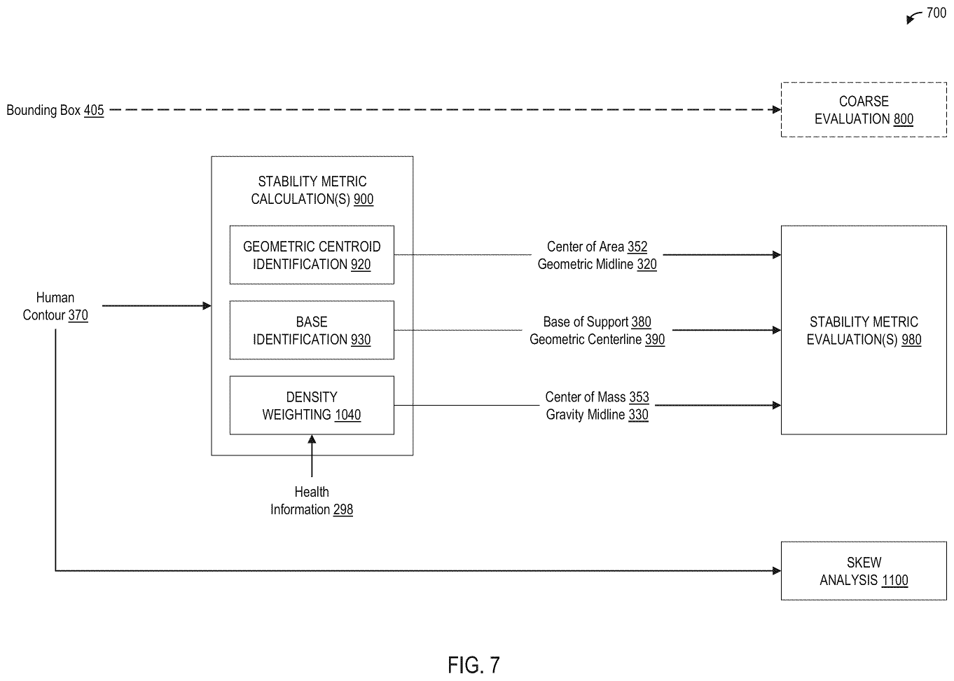

FIG. 2 is a high-level block diagram of a fall prevention process 200 performed by the fall prevention system according to exemplary embodiments. As shown in FIG. 2, the disclosed fall prevention process 200 includes a pose estimation process 400 (described in detail with reference to FIGS. 4-6) that uses the video images 224 captured by the image capture systems 120 to estimate the pose 270 of the user and a stability evaluation process 700 (described in detail with reference to FIGS. 7-12) to evaluate the stability of the user. In some embodiments, the fall prevention process 200 also includes a user identification process 210 (also described below). The fall prevention system generates feedback 280 for the user (e.g., auditory feedback output via the auditory feedback device 182 and/or haptic feedback output via the haptic feedback device 184) if, as a result of the stability evaluation 280, the system determines that the user is at risk of a fall.

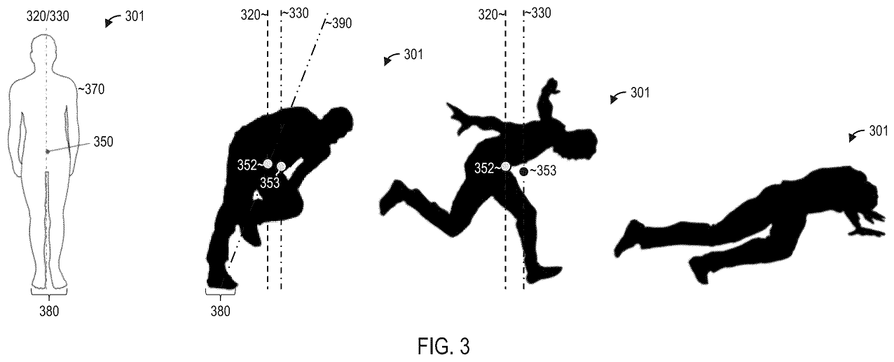

FIG. 3 are diagrams illustrating the human contour 370 of a user 301 identified by the pose estimation process 400 when the user 301 undergoes an unbalancing process leading to falling.

To determine when the user 301 deviates from the balance point (and provide feedback 280 to prevent a fall), various embodiments of the fall detection system identify metrics indicative of the stability of the user 301, including the center of gravity 350 of the user 301, the base of support 380 of the user 301, and the geometric centerline 390 of the user 301. The base of support 380 of the user 301 is the region of ground surface in contact with the human contour 370. The geometric centerline 390 of the user 301 is the line from the center of the base of support 380 of the user through the center of area of the body. The center of gravity 350 is the point at which the distribution of weight is the same in all directions given the gravitational field. (The center of gravity 350 of an erect user 301 with arms at the side is at approximately 56 percent of the height of the user 301 measured from the soles of the feet.) The center of gravity 350 shifts as the user 301 moves and bends. Because the act of balancing requires the maintenance of the center of gravity 350 above the base of support 380, stable posture is defined as having the center of gravity 350 placed within the boundaries of the base of support 380. According to the most recent research by biologists and physicians, a user 301 is more likely to fall when the human gravity centerline 340 deviates from base of support 380 and the angle between the geometric centerline 390 and the ground is less than a certain threshold. Therefore, accurate, real-time capture of the aforementioned metrics a fundamental challenges of fall prevention system.

As described below, the fall detection system may estimate the center of gravity 350 of the user by identifying the center of area 352 of the human contour 370 and/or estimating the center of mass 353 of the user 301. To evaluate the stability of the user 301, the fall detection system may also define a geometric midline 320 and/or a gravity midline 330 of the captured human contour 370. The geometric midline 320 is defined as the line parallel to the gravitational field through the center of area 352 of the human contour 370. The gravity midline 330 is defined as the line parallel to the gravitational field through the estimated center of mass 353 of the user 301.

FIG. 4 is a block diagram of various pose estimation processes 400 according to exemplary embodiments.

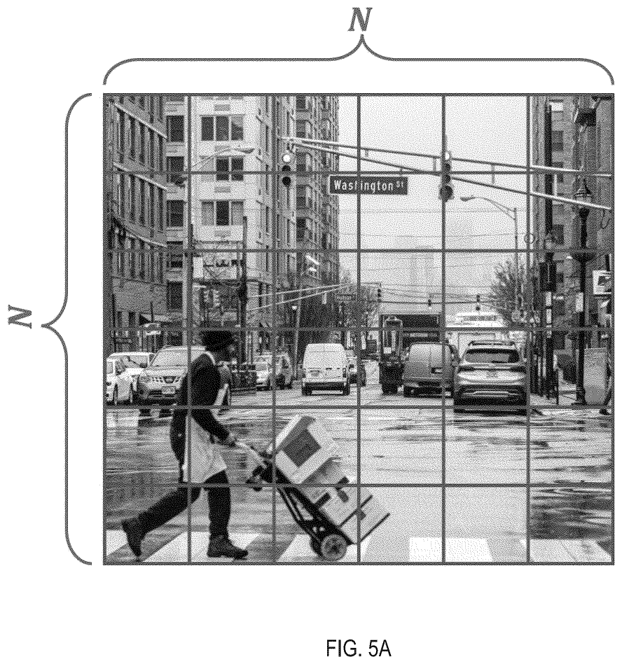

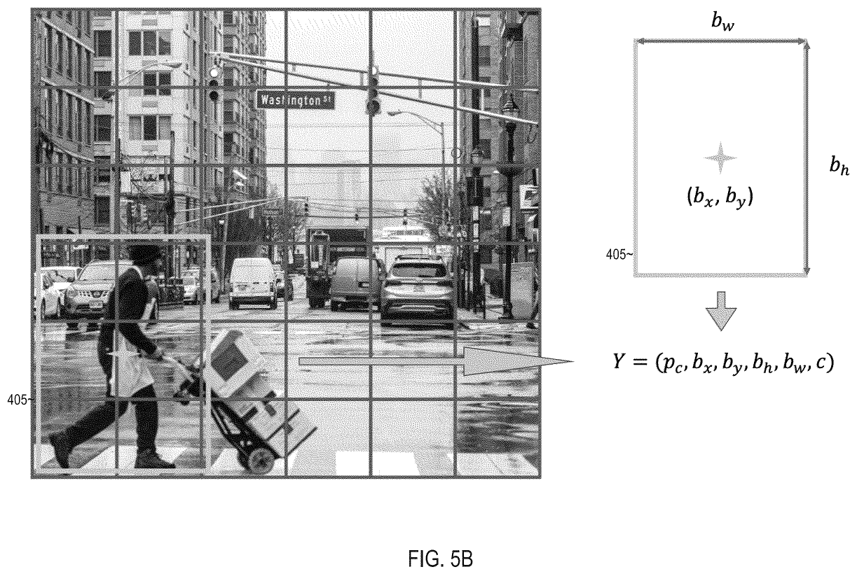

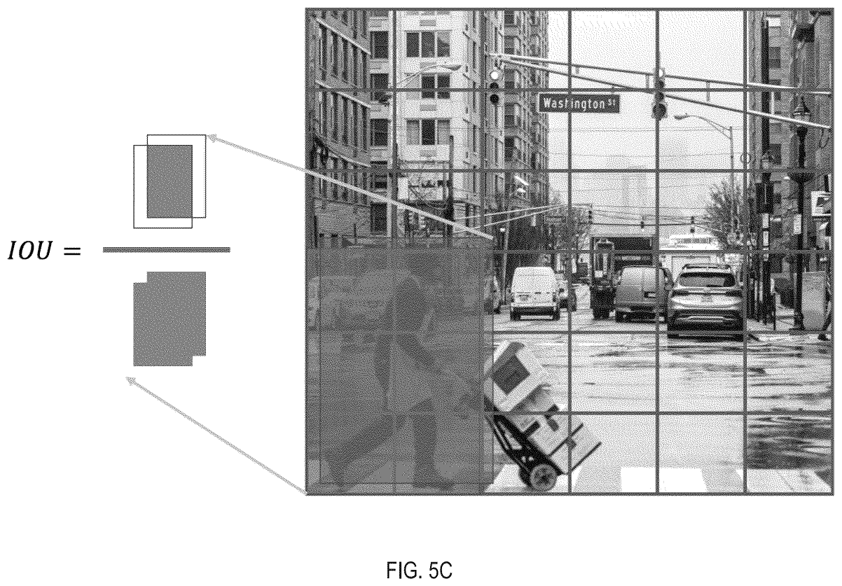

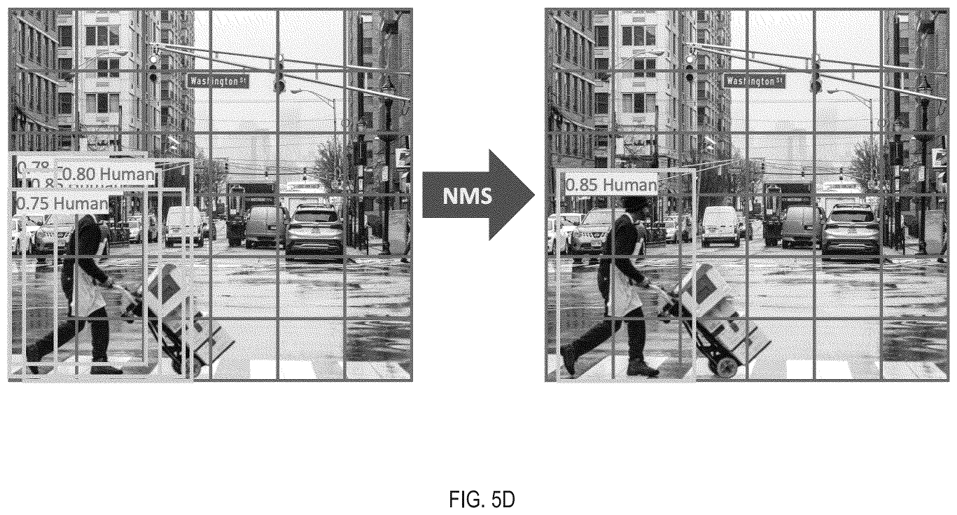

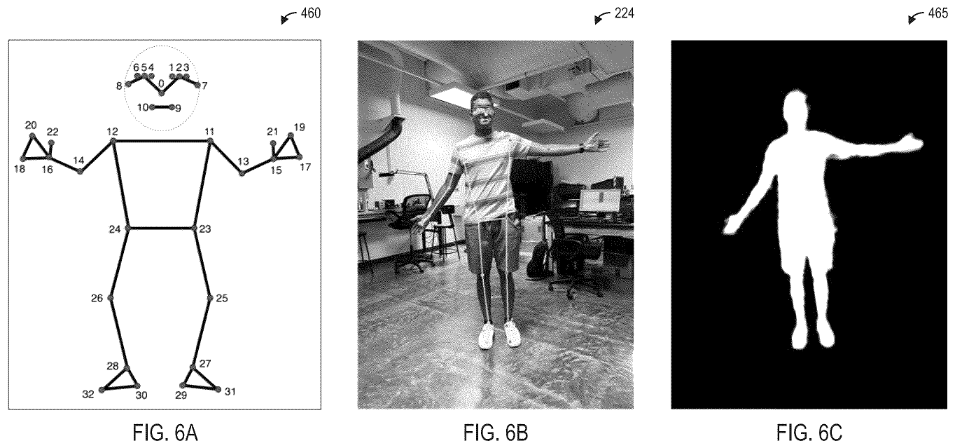

As shown in FIG. 4, the pose estimation process 400 estimates the human contour 370 of the user 370 based on the video images 224 (and, in some embodiments, depth information 226) received from the image capture systems 120. In some embodiments, the fall detection system includes a “back-to-front” pose estimation process 402, which includes a pose detection process 600 (described in detail below with reference to FIGS. 6A-6B) that identifies landmarks 460 within the image data 224 indicative of joints on the user 301 and an image segmentation process 650 (described in detail below with reference to FIG. 6C) that identifies a segmentation mask 465 indicative of the human contour 370 of the user 301. Additionally or alternatively, some embodiments of the fall detection system may include a “front-to-back” pose estimation process 401, including a body identification process 500 (described in detail below with reference to FIG. 5) that generates a bounding box 405 indicative of the location of the user 301 within the image data 224 and a background subtraction process 410 that identifies a silhouette 415 indicative of the human contour 370 of the user 301.

As briefly mentioned above, the body identification process 500 uses object detection algorithms to identify portions of the two-dimensional images 224 that include the user 301 and generates a bounding box 405 surrounding the portion of a two-dimensional image 224 that includes the user 301. The object detection algorithms applied by the system belong to you only look once (YOLO) algorithm family.

Generally speaking, YOLO builds on a series of maturely developed algorithms that employ convolutional neural networks (CNN) to detect objects in real-time. A CNN has input layers. The hidden layers conduct operations to discover data-specific characteristics. Convolution, Rectified linear unit (ReLU), and Pooling are the most common layers. Different features on an input image are activated after being filtered through the convolution layer. The process on ReLU, which is usually recognized as “activation,” carries the active features to the next layer. On the Pooling layer, the outputs are simplified thus reducing the amount of information that the network needs to learn. However, each CNN may contain 10,000 layers, with each layer learning to recognize a unique set of features. As a result, most of the time, the computational demands of running CNN are extreme. Moreover, CNN could be ineffective in encoding objects' position and orientation. That means if the object on the image is upside down, then CNN cannot accurately recognize the object. In addition, the accuracy of CNN is sensitive to adversarial factors; an insignificant fluctuation in inputs could alter the outputs of the network without a change visible to the human eye. Therefore, in our former work, we improve the efficiency of CNN by coupling it with YOLO algorithm family, which only requires a single run through the convolutional neural network to detect objects in real-time. YOLO is fast because it just requires a single CNN run per image. Moreover, YOLO observes the entire picture at once. This is a fundamental improvement to using CNN alone, which exclusively focuses on generated regions. The contextual information from the entire image, which prevents false positives, assists YOLO in overcoming the issues of encoding the location and orientation of the observables.

YOLO leverages CNN to identify different items quickly and accurately in an image in real-time. The algorithm accomplishes “object detection” as a regression problem, predicting a fixed number of quantities (the coordinates and the type of objects in terms of class probability) and only selecting the outputs with high confidence. For each image, the CNN is only required once for predicting multiple class probabilities and bounding boxes 405 simultaneously.

FIG. 5A illustrates an example original image divided into an N×N grid. The system uses the grid cells to locate a desired object and identify the located object. Probabilistic parameters will be utilized to tell the algorithm if the grid cell includes a desired object.

FIG. 5B illustrates an example of a rectangular-shaped bounding box 405 highlighting an object in the original image of FIG. 5A.

The system highlights all the objects in the original image using rectangular-shaped bounding boxes 405. In YOLO, each of the bounding boxes 405 is represented by a vector:

$$y = [ {p}_{c} , {b}_{x} , {b}_{y} , {b}_{h} , {b}_{w} , c ]$$

where p_{c} is the probability (scores) of the grid containing an object having class c; bₓ and b_{y} are the coordinate the center of the bounding box; bₕ and b_{w} are the height and the width of the bounding box with respect to the enveloping grid cell; and c is the class of the objects.

FIG. 5C illustrates Intersection Over Unions (IOU), a parameter for distinguishing grids highly relevant to the objects from lowly ones. The expression of IOU is:

$$IOU = \frac{area of the intersection between grid and bounding box}{area of the union between grid and bounding box}$$

The system compares the calculated IOU to predetermined threshold and discards the grid cell if its IOU is lower than the predetermined threshold.

FIG. 5D illustrates the use of Non-Maximum Suppression (NMS) to keep the bounding boxes 405 with the highest probability scores. Keeping all the bounding boxes 405 may produce noise when an object has several boxes with a high IOU. Accordingly, the system may employ Non-Maximum Suppression (NMS) to keep the bounding boxes 405 with the highest probability (scores).

Referring back to FIG. 4, the background subtraction process 410 identifies the silhouette 415 of the user 301 by removing portions of the video image 224 that show background objects and a polygon in the shape of the remaining image data 224. The background subtraction process 410 may be performed, for example, using the BackgroundSubtractor function included in the OpenCV library. To identify image data 224 depicting background objects (and, by extension, distinguish between image data 224 depicting background objects and image data 224 depicting the user 301), the background subtraction algorithm 410 (e.g., BackgroundSubtractor) may be trained using images of the environment 101 without the user 301. Having been trained using images of the environment 101 without the user 301, the background subtraction algorithm recognizes image data 224 depicting objects (such as the user 301) that are not part of the learned environment.

By subtracting the image data 224 depicting background objects, a silhouette 415 indicative of the user 301 is obtained. Because the contours of the silhouette 415 obtained by the background subtraction algorithm 410 may be rough and inaccurate, the background subtraction algorithm 410 may also use color information included in the image data 224 (and, in some embodiments depth information 226 captured by the image capture system 120) to refine the silhouette 415 and form a version that more accurately depicts the human contour 370 of the user 301.

In some embodiments, the fall detection system may estimate the human contour 370 of the user 301 using pose detection 600 and image segmentation 650. The pose detection 600 and image segmentation 650 processes may be performed, for example, using a pre-trained machine learning model for human pose estimation (for example, algorithms included in Mediapipe Pose, which are rapidly deployable python API applications from the TensorFlow-based Mediapipe Open Source Project). The pose detection 600 and image segmentation 650 processes (e.g., included in Mediapipe Pose) infer landmarks 460 (i.e., estimated locations of joints of the user 301) and a segmentation mask 465 (i.e., the estimated human contour 370 of the user 301) from the RGB image frames 224.

FIG. 6A is a diagram of the landmarks 460 (identified in Table 1 below) generated by the pose detection process 600 according to exemplary embodiments. FIG. 6B is an example image 224 with the landmarks 460. FIG. 6C is an example segmentation mask 465 identified by the image segmentation process 650 based on the example image 224 of FIG. 6B and the landmarks 460 identified using the pose detection process 600.

TABLE 1

| | |
|---|---|
| 0 | nose |
| 1 | left eye (inner) |
| 2 | left eye |
| 3 | left eye (outer) |
| 4 | right eye (inner) |
| 5 | right eye |
| 6 | right eye (outer) |
| 7 | left ear |
| 8 | right ear |
| 9 | mouth (left) |
| 10 | mouth (right) |
| 11 | left shoulder |
| 12 | right shoulder |
| 13 | left elbow |
| 14 | right elbow |
| 15 | left wrist |
| 16 | right wrist |
| 17 | left pinky |
| 18 | right pinky |
| 19 | left index |
| 20 | right index |
| 21 | left thumb |
| 22 | right thumb |
| 23 | left hip |
| 24 | right hip |
| 25 | left knee |
| 26 | right knee |
| 27 | left ankle |
| 28 | right ankle |
| 29 | left heel |
| 30 | right heel |
| 31 | left foot index |
| 32 | right foot index |

Obtaining the human contour 370 using pose detection 600 and image segmentation 350 provides specific benefits when compared to systems that rely solely on body identification 500 and background subtraction 410. Body identification 500 and background subtraction 410 algorithms are sensitive to light and dependent to the precision of the depth information 226. By contrast, the pose detection 600 and image segmentation 650 algorithms apply a segmentation mask 465 directly to the image data 224 depicting the user 301 without interacting with the image data 224 depicting the environment 101, minimizing the sensitivity to environmental complexities such as light fluctuations.

Current pose detection 600 and image segmentation 650 algorithms (e.g., the TensorFlow Lite versions of Mediapipe Pose) are highly computationally efficient as compared to current body identification 500 and background subtraction 410 algorithms. Meanwhile, pose detection 600 and image segmentation 650 can identify the human contour 265 without the need for body identification 500 and background subtraction 410. Accordingly, some embodiments of the fall detection system may rely solely on pose detection 600 and image segmentation 650 (and may not include the body identification 500 and background subtraction 410) processes to reduce computational expense. However, as body identification 500 and background subtraction 410 algorithms are further developed, those processes may become more efficient than the pose detection 600 and image segmentation 650 that are available. Accordingly, to take advantage of the most accurate and computationally effective methods available, the fall detection system can be configured to use either (or both) of the front-to-back or back-to-front pose estimation process 401 and 402 described above.

The pose estimation process 400 is performed individually for each stream of video images 224 received from each image capture system 120. Accordingly, using either or both of the processes 401 and 402 described above, the fall prevention system captures a two-dimensional silhouette 415 and/or segmentation mask 465 indicative of the human contour 370 of the user 301 from the point of view of the image capture system 120 providing the video images 224. In some embodiments, the silhouette 415 and/or segmentation mask 465 from the point of view of one image capture system 120 may be refined using image data 224 captured by another image capture system 120. For example, image data 224 captured from multiple angles may be overlayed to refine the contours of the captured silhouette 415 and/or segmentation mask 465. In other embodiments, the silhouette 415 and/or segmentation mask 465 from the point of view of that image capture system 120 may be identified using the video images 224 received only from that image capture system 120.

In embodiments where the image capture system 120 also captures depth information 226, a depth incorporation process 470 may be performed to incorporate the captured depth information 226 into the human contour 370 of the user 301 from the point of view of that image capture system 120. For example, the captured human contour 370 may include both the captured two-dimensional silhouette 415 and/or segmentation mask 465 and the depth of each pixel of the captured two-dimensional silhouette 415 and/or segmentation mask 465.

FIG. 7 is a block diagram of various stability evaluation processes 700 according to exemplary embodiments.

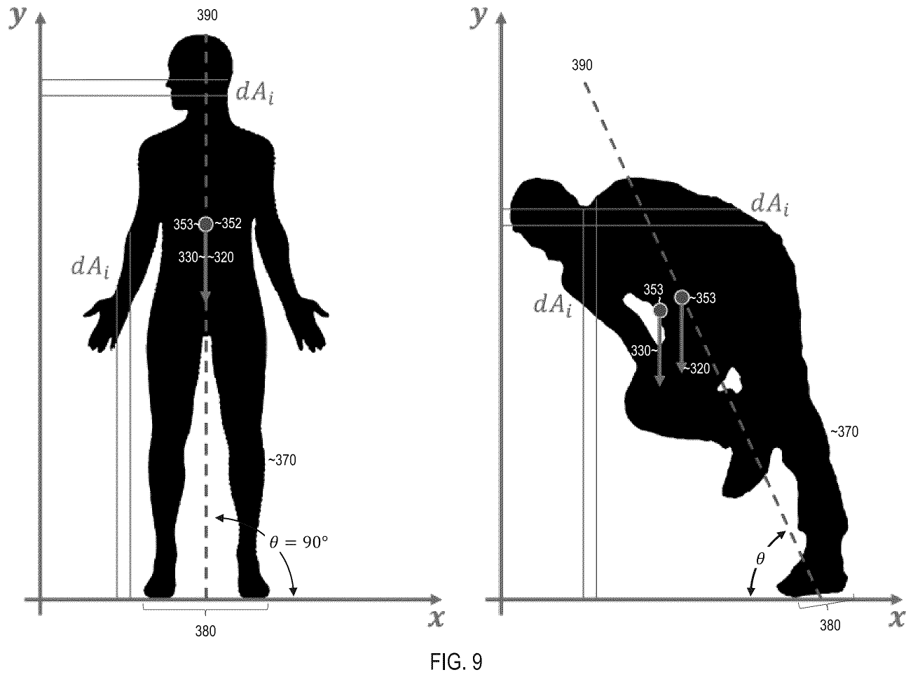

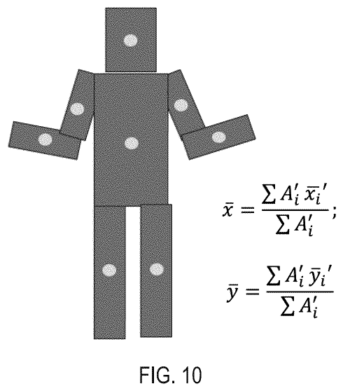

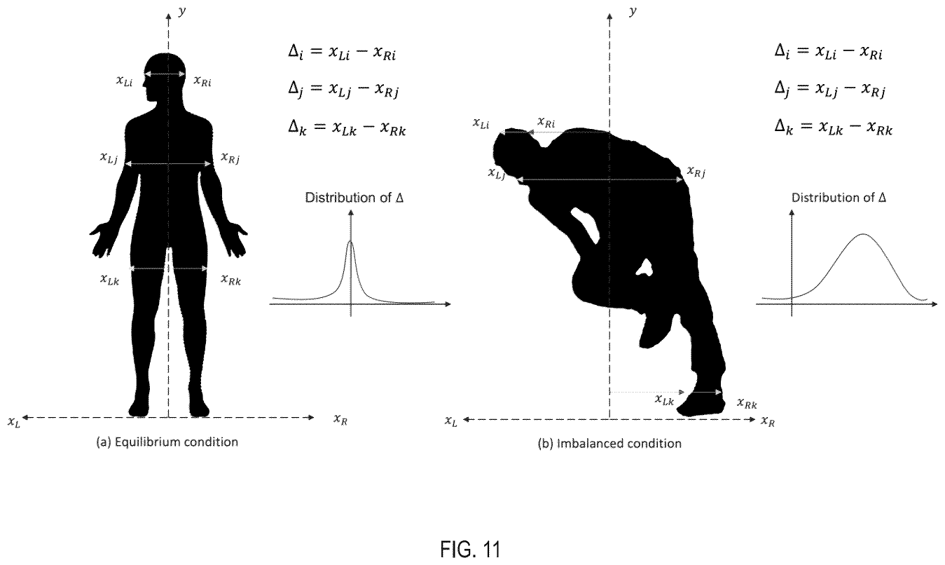

As shown in FIG. 7, the various stability evaluation processes 700 may include stability metric calculations 900 and stability metric evaluations 980 (described in detail below with reference to FIGS. 9 and 10) and/or a skew analysis 1100 (described in detail below with reference to FIG. 11). The stability metric calculations 900 may include geometric centroid identification 920 (described in detail below with reference to FIG. 9) to identify the center of area 352 and the geometric midline 320 of the captured human contour 370, a base identification process 930 to identify the base of support 380 and the geometric centerline 390 of the captured human contour 370 (also described in detail below with reference to FIG. 9), and/or a density estimation process 1040 (described in detail below with reference to FIG. 10) to estimate the center of mass 353 and the gravity midline 330 of the user 301.

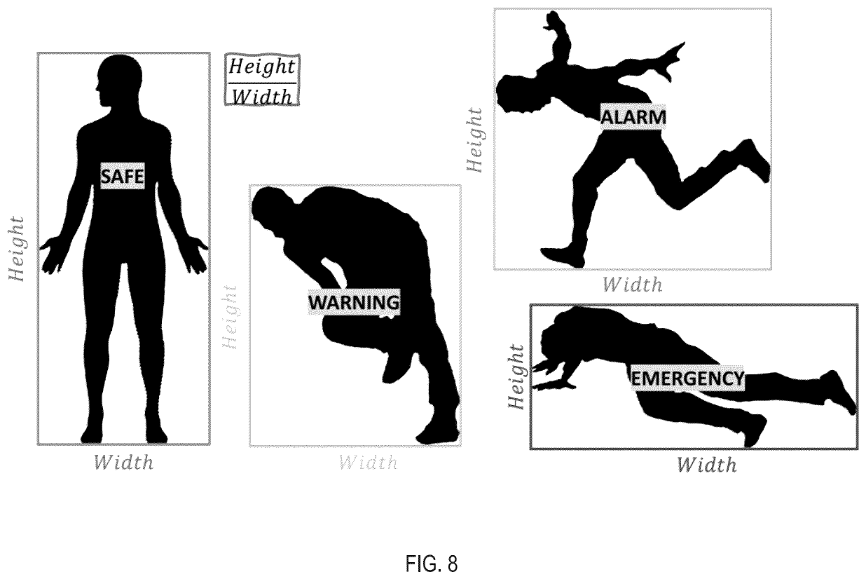

In embodiments of the fall detection system that identify a bounding box 405 surrounding image data 224 that includes the user 301, the fall prevention system may also perform a course stability evaluation 800 (described in detail below with reference to FIG. 8), for example to quickly alert the user of a potential problem even before a more precise stability evaluation can be performed.

FIG. 8 is an illustration of example course stability evaluations 800 according to an exemplary embodiment.

As briefly mentioned above, embodiments of the fall detection system that identify a bounding box 405 surrounding image data 224 of the user 301 may first perform a course stability evaluation 800 based on the dimensions of the bounding box 405 identified by the body identification process 500. If the human body is depicted as a rectangular box, the height-to-width ratio of this rectangular box is significantly changed when a person falls. Accordingly, the fall detection system may provide feedback 280 via the feedback device 180 when the height-to-width radio is smaller than a predetermined threshold (e.g., 1.0).

FIG. 9 is a diagram of stability metrics generated using the human contour 370 of a stable user 301 and an unstable user 301 according to exemplary embodiments.

As briefly mentioned above, one estimate of the center of gravity 350 of the user 301 may be determined by assuming the density of the body is uniform and calculating the center of area 352 (x̅, y̅) of the captured two-dimensional human contour 370 as follows:

$$\overline{x} = \frac{{\int }_{{x}_{min}}^{{x}_{max}} dA * x}{A}$$

$$\overline{y} = \frac{{\int }_{{y}_{min}}^{{y}_{max}} dA * y}{A}$$

Meanwhile, the geometric midline 320 may be defined as the line parallel to the gravitational field through the center of area 352.

The stability metrics may also include the base of support 380 and the geometric centerline 390 of the captured human contour 370. In embodiments that use pose estimation 600 to capture a segmentation mask 465, the base of base of support 380 may be identified based on the landmarks 460 indicative of the toes, feet, and heels. (Additionally, when there is no contact between the feet of the user 301 and the ground, the fall detection system includes activity detection algorithms that detect contact between human body and other supporting surfaces, such as a chair, a bed, a wall, etc.) In embodiments that use background subtraction 410 to capture a silhouette 415, the base of base of support 380 may be identified by identifying the interface between the user 301 and the ground at the moment the image data 224 of the user 301 is separated from image data 224 of the background environment. (Additionally, depth information 226 may be used to refine the estimate of the location of the base of support 380.) Meanwhile, the geometric centerline 390 may be calculated by identifying the line extending from the center of the base of support 380 through the center of area 352 of the captured human contour 370.

The stability metrics may also include the center of mass 353 and the gravity midline 330 of the user 301. As briefly mentioned above, the center of gravity 350 is the point at which the distribution of weight is the same in all directions given the gravitational field. If the density of the body is uniform, the center of gravity 350 can be accurately estimated by finding the center of area 352 of the captured human contour 370 as described above. However, because the density of the human body is not uniform, the center of gravity 350 of the user 301 can be more accurately identified by using combining the captured human contour 370 and health information 298 of the user 301 (e.g., the height and weight of the user 301) to estimate the center of mass 353 of the user 301.

FIG. 10 is a diagram illustrating a process for estimating the center of mass 353 of the user 301 according to exemplary embodiments.

In some embodiments, the fall detection system may estimate the density of each body part included in the captured two-dimensional human contour 370 (e.g., based on the height and weight of the user 301) and estimate the center of mass 452 (x̅, y̅) of the captured human contour 370 as follows:

$$\overline{x} = \frac{\int {\int }_{R} x ρ ( x , y ) dA}{\int {\int }_{R} ρ ( x , y ) dA}$$

$$\overline{y} = \frac{\int {\int }_{R} y ρ ( x , y ) dA}{\int {\int }_{R} ρ ( x , y ) dA}$$

where ρ(x, y) is the density of the body at point (x, y) and R is the region within the body outline.

Alternatively, to improve computational efficiency and provide feedback 280 in near real time, the fall detection system may assign simple geometric shapes (e.g., rectangles) to a wireframe indicative of the captured human contour 370 (e.g., a wireframe connecting the landmarks 460) as shown in FIG. 10, estimate the density of each shape geometric based on health information 298 of the user 301 (e.g., the height and weight of the user 301), and use those formulas estimate the center of mass 452 (x̅, y̅) of the captured human contour 370.

As shown in FIG. 7, the fall detection system performs stability metric evaluation(s) 980 to determine whether the user 301 is likely to fall and, if so, output feedback 280 to the user 301. As shown in FIG. 9, for a perfectly stable user 301, the gravity midline 330 is within the horizontal boundaries of the base of support 380 and the geometric centerline 390 forms a 90-degree angle θ with the ground. Additionally, the center of area 352 of the captured human contour 370 may be coincident with the center of mass 353 of the user 301 (and, by extension, the geometric midline 320 may be coincident with the gravity midline 330). As a user 301 becomes unstable, however, the gravity midline 330 may deviate from the horizontal boundaries of the base of support 380, the angle θ between the geometric centerline 390 and the ground decreases, and the center of area 352 (and the geometric midline 320) of the captured human contour 370 may deviate from the center of mass 353 of the user 301 (and the gravity midline 330). Accordingly, in various embodiments, the fall detection system may determine that the user 301 is likely to fall (and output feedback to the user 301), for example, if the gravity midline 330 deviates from the horizontal boundaries of the base of support 380, if the angle θ between the geometric centerline 390 and the ground is less than 90 degrees by more than a predetermined threshold (or if the angle between the geometric centerline 390 and either the geometric midline 320 or the gravity midline 330 deviates from 0 degrees by more than the predetermined threshold), and/or if the center of area 352 (or the geometric midline 320) of the captured human contour 370 deviates from the center of mass 353 (or the gravity midline 330) of the user 301.

In some embodiments, the fall detection system may determine that the user 301 is likely to fall (and output feedback 280 to the user 301) based on third order moment (i.e., the skewness).

FIG. 11 illustrates a calculation of the skewness according to exemplary embodiments. The y axis is defined as the line passing through the human gravity center and being perpendicular to the ground; the two horizontal axes are defined as the axes originated at the projection of the human gravity center on the ground and pointing in opposite directions. As shown in FIG. 11, the system may calculate the symmetry of the body using the centerline 240 relative to the edge/outline of the human contour 370. As shown in FIG. 11, skewness may be calculated by the summing the horizontal vectors from the centerline 240 to the edge/outline of the human contour 370 at various heights (e.g., three heights as shown in FIG. 11). In the equilibrium condition, the sum of each vector is 0. In the imbalanced condition, however, the sum of some or all of the vectors will have a magnitude greater than 0.

To more accurately estimate the three-dimensional pose 270 of the user 301 in three-dimensional space, some embodiments of the fall detection system may perform a three-dimensional reconstruction of the three-dimensional human contour 370 using image data 224 and/or depth information 226 captured by multiple image capture systems 120.¹ In those embodiments, the fall detection system may perform a single stability evaluation 700 of the reconstructed three-dimensional human contour 370. ¹ In those embodiments, the three-dimensional human contour 370 may be constructed as a volumetric occupancy grid, which represents the state of the environment as a three-dimensional lattice of random variables (each corresponding to a voxel) and a probabilistic estimate of the occupancy of each voxel as a function of incoming sensor data and prior knowledge. Occupancy grids allow for efficient estimates of free space, occupied space, and unknown space from range measurements, even for measurements coming from different viewpoints and time instants. A volumetric occupancy grid representation is richer than those which only consider occupied space versus free space, such as point clouds, as the distinction between free and unknown space can potentially be a valuable shape cue. Integration of a volumetric occupancy grid representation with a supervised 3D CNN has been shown to be effective in object labeling and classification even with background clutter (See Maturana, D. and Scherer, S., 2015, September. Voxnet: A 3d convolutional neural network for real-time object recognition. In 2015 IEEE/RSJ international conference on intelligent robots and systems (IROS) (pp. 922-928). IEEE.)

To provide feedback 280 in real time, however, three-dimensional reconstruction may require more processing time (and/or more processing power) than is available. Accordingly, as shown in FIG. 2, the fall detection system may individually perform the pose estimation 400 and the stability evaluation 700 processes described above using the video images 224 (and, in some embodiments, depth information 226) captured by each image capture system 120. In those embodiments, the fall detection system may output feedback 280 to the user 301 if the stability evaluation 700 of any estimated pose 270 of the user 301 (from the point of view of any of the image capture systems 120) indicates that the user 301 may be likely to fall.

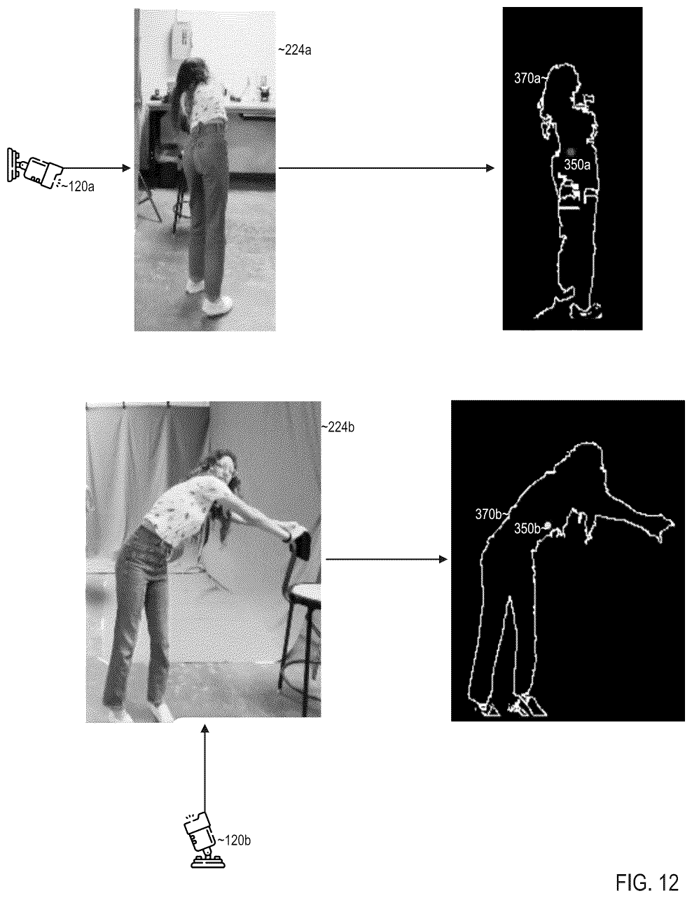

FIG. 12 illustrates how the use of multiple image capture systems 120a and 120b by the fall detection system can more accurately determine whether the user 301 is likely to fall.

The example of FIG. 12 includes image data 224a of a user 301 captured at a first angle by a first image capture system 120a, the human contour 370a and the center of gravity 350a of the user 301 from the point of view of the first image capture system 120a, image data 224b of a user 301 captured at a second angle by a second image capture system 120b, and the human contour 370b and center of gravity 350b of the user from the point of view of the second image capture system 120b. As shown in FIG. 12, relying only on image data 224a from the point of view of the first image capture system 120a may lead to an incorrect determination that the user 301 is in a stable pose. However, by using multiple image capture system 120 to capture image data 224 of the user 301 from multiple angles, the fall detection system can more accurately determine (in the example of FIG. 12, using the image data 224b captured by the second image capture system 120b), that the user 301 is, in fact, in a potentially unstable pose.

When multiple humans exist in a certain space, the fall detection system may be configured to distinguish the user 301 from other occupants. Referring back to FIG. 2, for example, the fall prevention system may include a user identification process 210 that identifies video images 224 depicting the user 301. In those embodiments, the fall prevention system may only perform the pose estimation 400 and stability evaluation 700 processes using video images 224 of the user 301. In other embodiments (for example, to address the privacy concerns inherent in user identification), the fall prevention system may not perform the user identification process 210 (and may, instead, output feedback 280 in response to a determination that any human in the environment 101 is may be likely to fall). The fall detection system also protects the privacy of users 301 and other individuals by using the video images 224 for the sole purpose of identifying the human contour 370 as described above without storing those video images 224 for longer than is necessary to identifying the human contour 370.

Referring back to FIG. 1B, the fall prevention process 200 may be realized as software instructions stored and executed by the server 160. However, to provide feedback 280 in real time, in preferred embodiments the fall prevention process 200 is realized by software instructions stored and executed by the local controller 190. For instance, the local controller 190 may store and execute the pretrained machine learning models described above, which may be received from (and, in some instances, updated by) the server 160.

As briefly mentioned above, the local controller 190 may be integrated into the feedback device 180 (as shown in FIG. 1B) or may be realized as a separate device—for example, a wearable computing device, a personal computer, an application-specific hardware device (e.g., such as an application-specific integrated circuit or other controller), etc.—that communicates with the feedback device 180 via a wired or wireless (direct or network) connection. In order to perform the functions described above and provide feedback 280 quickly enough to provide prevent falls, in preferred embodiments the local controller 180 receives the video images 224 from the image capture devices 120 via a local area network 172 or other local connection (as opposed to a wide area network 178 such as the Internet). Accordingly, in preferred embodiments, the local controller 180 is located within the environment 101 of the user 301 or sufficiently close to it (e.g., within the same facility) so as to receive the video images 224 from the image capture systems 120, process those video images 224 as described above, and transmit instructions to the feedback device 180 in a time period that is sufficiently short enough to provide feedback 280 in near real-time (and, ideally, detect a potential fall and alert the user before the fall occurs).

As used herein, a “local area network” may include any number of networks used by hardware computing devices located within the environment 101 of the user using any number of wired and/or wireless protocols. For example, the local area network 172 may include both a local network utilizing both wireless (e.g., WiFi) and/or wired connections (e.g., Ethernet) and hardware devices communicating directly via wired connections (e.g., USB) and/or wireless connections (e.g., Bluetooth). The environment 101 of the user 301 may include any environment in which the disclosed fall detection system is used to monitor the user 301 and provide feedback 280 as described above. For example, the environment 101 of the user 301 may be the user's home or workplace, a personal care facility, a hospital, etc.

When synchronizing multiple image capture systems 120, the performance of real-time updates will possibly be hindered due to insufficient computing power. Accordingly, the preferred embodiments of the disclosed system employ the Mediapipe pose estimator accompanied by the integration of the Mediapipe-based object detection library and face recognition package. That integration ensures that the system's algorithm is constructed using the TensorFlow model and effectively addresses the computational cost associated with compatibility issues right from the outset. Moreover, preferred embodiments employ parallel computing techniques, such as multiprocessing, that apply peripheral CPU cores to reduce the computational demands to execute the pose detection process 600.

The disclosed system can be combined with the system of U.S. patent application Ser. No. 18/236,842, which provides users with audio descriptive objects in their environment. That feature is critically important, especially when changes in visual perception occur (temporarily or permanently) and prevent users from colliding with surrounding objects. It is understood that high glucose can change fluid levels or cause swelling in the tissues of eyes triggering focus distortion and blurred vision. Focus distortion and blurred vision could take place temporarily or become a long-lasting problem. Accordingly, the disclosed system can identify and inform users if they get too close to objects on the floor.

While preferred embodiments have been described above, those skilled in the art who have reviewed the present disclosure will readily appreciate that other embodiments can be realized within the scope of the invention.

What is claimed is:

1. A fall prevention method, comprising:
receiving, via a local area network by a local controller in an environment of a user, video images of the user from each of a plurality of image capture systems in the environment of the user;
storing, by the local controller, one or more pre-trained machine learning models for estimating a pose of the user;
using the one or more pre-trained machine learning models, by the local controller, to capture at least one human contour indicative of the pose of the user based on the video images received from each of the plurality of image capture systems;
determining, for each captured human contour, whether the captured human contour is indicative of an unstable pose; and
outputting audible or haptic feedback to the user in response to a determination that a captured human contour is indicative of an unstable pose.

2. The method of claim 1, wherein capturing at least one human contour based on the video images received from each of the plurality of image capture systems comprises capturing, for each of the plurality of image capture systems, a two-dimensional human contour indicative of the pose of the user from the point-of-view of the image capture system.

3. The method of claim 2, further comprising:
receiving depth information from each image capture system; and
identifying the depth of each pixel of each captured two-dimensional human contour.

4. The method of claim 3, wherein each image capture system comprises a depth camera or light detection and ranging (LiDAR) scanner.

5. The method of claim 2, wherein audible or haptic feedback is output in response to a determination that any two-dimensional human contour from the point-of-view of any of the image capture systems is indicative of an unstable pose.

6. The method of claim 1, wherein capturing at least one human contour based on the video images received from each of the plurality of image capture systems comprises reconstructing a three-dimensional human contour indicative of the three-dimensional pose of the user based on the video images received from the plurality of image capture systems.

7. The method of claim 1, wherein capturing the at least one human contour using the one or more pre-trained machine learning models comprises:
using a pre-trained pose detection model to infer landmarks indicative of joints of the user; and
using a pre-trained image segmentation model to infer a segmentation mask indicative of the pose of the user.

8. The method of claim 1, wherein capturing the at least one human contour using the one or more pre-trained machine learning models comprises:
training a background subtraction model to identify image data depicting the environment;
using a pre-trained body identification model to identify a bounding box surrounding image data depicting the user; and
using the trained background subtraction model to subtract image data depicting the environment from the image data within the bounding box.

9. The method of claim 7, wherein the bounding box has a height and a width and the determination of whether the captured human contour is indicative of an unstable pose is based on a comparison of the height and the width of the bounding box.

10. The method of claim 1, wherein determining whether the captured human contour is indicative of an unstable pose comprises:
identifying a base of support of the user;
estimating a center of mass of the user;
identifying a gravity midline extending perpendicular to the gravitational field from the estimated center of mass of the user; and
determining whether the gravity midline is within the base of support of the user.

11. The method of claim 10, wherein estimating the center of mass of the user comprises:
storing health information of the user;
estimating, based on the health information of the user, the density of one or more body parts of the user; and
estimating the center of mass of the user based on the captured human contour and the estimated density of each of the one or more body parts of the user.

12. The method of claim 11, wherein the health information includes height and weight and the density of the one or more body parts of the user are estimated based on the height and weight of the user.

13. The method of claim 11, wherein estimating the center of mass of the user comprises:
assigning geometric shapes to a wireframe indicative of the pose of the user;
estimating the density of each geometric shape based on the health information of the user; and
estimating the center of mass of the geometric shapes indicative of the pose of the user.

14. The method of claim 1, wherein determining whether the captured human contour is indicative of an unstable pose comprises:
identifying a base of support of the user;
identifying a center of area of the captured human contour;
identifying a geometric midline extending from the center of the base of support of the user through the center of area of the captured human contour; and
determining whether the captured human contour is indicative of an unstable pose based on an angle of the geometric midline.

15. The method of claim 1, wherein determining whether the captured human contour is indicative of an unstable pose comprises:
identifying a center of area of the captured human contour;
estimating a center of mass of the user; and
determining whether the captured human contour is indicative of an unstable pose based on a distance between the center of area of the captured human contour and the estimated center of mass of the user.

16. A fall prevention system, comprising:
a plurality of image capture systems in an environment of a user;
a local controller, in communication with the plurality of image capture systems via a local area network, that:
stores one or more pre-trained machine learning models for estimating a pose of the user;
receives video images of the user from each of the plurality of image capture systems;
uses the one or more pre-trained machine learning models to capture at least one human contour indicative of the pose of the user based on the video images received from each of the plurality of image capture systems; and
determines, for each captured human contour, whether the captured human contour is indicative of an unstable pose; and
a feedback device that outputs audible or haptic feedback to the user in response to a determination that a captured human contour is indicative of an unstable pose.

17. The system of claim 16, wherein, for each of the plurality of image capture systems, the local controller captures a two-dimensional human contour indicative of the pose of the user from the point-of-view of the image capture system.

18. The system of claim 17, wherein the feedback device outputs feedback in response to a determination that any two-dimensional human contour from the point-of-view of any of the image capture systems is indicative of an unstable pose.

19. The system of claim 16, wherein the local controller captures the at least one human contour by:
using a pre-trained pose detection model to infer landmarks indicative of joints of the user; and
using a pre-trained image segmentation model to infer a segmentation mask indicative of the pose of the user.

20. The system of claim 16, wherein the local controller captures the at least one human contour by:
using a pre-trained body identification model to identify a bounding box surrounding image data depicting the user; and
using a background subtraction model that has been trained to identify image data depicting the environment to subtract image data depicting the environment from the image data within the bounding box.
